(19) United States
(12) Patent Application Publication
(10) Pub. No.: US 2017/0232264 A1
(43) Pub. Date: Aug. 17, 2017

(54) MICRO EXTENSION CONNECTOR FOR ELECTRICAL STIMULATION SYSTEMS

(71) Applicant: Boston Scientific Neuromodulation Corporation, Valencia, CA (US)

(72) Inventor: Joshua Dale Howard, Sacramento, CA (US)

(21) Appl. No.: 15/430,059

(22) Filed: Feb. 10, 2017

Related U.S. Application Data
(60) Provisional application No. 62/294,907, filed on Feb. 12, 2016.

(51) Int. Cl. A61N 1/375; A61N 1/05

(52) U.S. Cl. CPC A61N 1/3752; A61N 1/05

(57) ABSTRACT

An electrical stimulation system includes a lead having lead conductors, electrodes, a lead extension having conductors, and terminals. The system includes a connector having a first and second connection elements that each have sealing members and contacts at least partially disposed within the sealing members. The lead and lead extension are coupled to one of the connection elements, respectively, and the contacts are electrically coupled to lead or lead extension conductors. The connection elements electrically couple the lead conductors with the lead extension conductors. Lastly, the connector includes a coupler engageable with the connection elements to urge the respective sealing members together to resist bodily fluid interaction with the conductors and contacts.

### CROSS-REFERENCE TO RELATED APPLICATIONS

This application claims the benefit under 35 U.S.C. §119(e) of U.S. Provisional Patent Application Ser. No. 62/294,907, filed Feb. 12, 2016, which is incorporated herein by reference.

### FIELD

The present invention is directed to the area of implantable electrical stimulation devices and methods of making and implanting the same. The present invention is also directed to a micro extension connector for use within a lead.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, an electrical stimulation system includes a lead having a plurality of lead conductors and a plurality of electrodes located along a distal portion of the lead in which the electrodes are electrically coupled to the plurality of lead conductors. The electrical stimulation system further includes a lead extension having a plurality of lead extension conductors and a plurality of terminals located along a proximal portion of the lead extension in which the terminals are electrically coupled to the lead extension. The electrical stimulation system further includes a connector having a first and second connection elements. The first connection element includes a first sealing member and a plurality of first contacts at least partially disposed within the first sealing member. The second connection element includes a second sealing member and a plurality of second contacts at least partially disposed within the second sealing member. The lead is coupled to one of the first or second connection elements and the plurality of first contacts are electrically coupled to a one of the lead conductors or the lead extension conductors. Likewise, the lead extension is coupled to another one of the first or second connection elements and the plurality of second contacts are electrically coupled to another one of the lead conductors or the lead extension conductors. In at least some embodiments, the first and second connection elements electrically couple the lead conductors with the lead extension conductors. Lastly, the connector includes a coupler engageable with the first and second connection elements to urge the first and second sealing members together to resist bodily fluid interaction with the lead conductors, the lead extension conductors, the first contacts, or the second contacts.

In at least some embodiments, the electrical stimulation system includes a central lumen extending through the first and second sealing members. In at least some embodiments, the first and second housings include complementary keying features. In at least some embodiments, at least one of the first or second contacts can be crimpable contacts. In at least some embodiments, the first and second housings may be free to rotate relative to the first and second sealing members, respectively. In at least some embodiments, the first and second sealing members can be made from an elastomeric material. In at least some embodiments, the coupler includes wing portions that extend from the coupler and at least one of the wing portions includes a suture hole.

In at least some embodiments, the electrical stimulation system includes a control module having a housing and an electronic subassembly disposed in the housing, wherein the lead extension is electrically coupleable to the electronic subassembly.

In another embodiment, a connector includes a first connection element having a first housing and a first non-conductive, elastomeric sealing member at least partially disposed within the first housing, wherein the first housing includes a first outer surface and first machine threads formed on the first outer surface. The connector includes a plurality of male contacts at least partially disposed within the first sealing member. The connector includes a second connection element having a second housing and a second, non-conductive, elastomeric sealing member extending from the second housing, wherein the second housing includes a second outer surface and second machine threads formed on the second outer surface. The connector includes a plurality of female contacts at least partially disposed within the second sealing member. And, the connector includes a coupler having a first end portion and a second end portion, the first end portion having first internal threads for engaging the first machine threads, the second end portion having second internal threads for engaging the second machine threads, wherein engagement of the coupler with the first and second connection elements urges the first and second sealing members together to resist bodily fluid from interacting with the male contacts, the female contacts, or both.

In at least some embodiments, the first housing is free to rotate relative to the first sealing member. In at least some embodiments, the second housing is free to rotate relative to the second sealing member. In at least some embodiments, the connector further includes a central lumen extending through the first and second sealing members. In at least some embodiments, the first and second housings include complementary keying features. In at least some embodiments, the first and second sealing members are made from a polymeric material. In at least some embodiments, the coupler includes wing portions that extend from the coupler. In at least some embodiments, at least one of the wing portions includes a suture hole.

In another embodiment, a method of stimulating patient tissue includes the steps of (1) implanting the electrical stimulation system described above into the patient tissue and (2) controllably modulating the plurality of electrodes with electrical pulses from a control module to stimulate the patient tissue.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a lead electrically coupled to a control module according to an embodiment of the present invention;

FIG. 2A is a schematic view of one embodiment of the control module of FIG. 1 configured and arranged to electrically couple to an elongated device according to an embodiment of the present invention;

FIG. 2B is a schematic view of one embodiment of a lead extension configured and arranged to electrically couple the elongated device of FIG. 2A to the control module of FIG. 1 according to an embodiment of the present invention;

FIG. 3 is a schematic perspective view of a connector according to an embodiment of the present invention;

FIG. 4A is a schematic, perspective view of a first connection element according to an embodiment of the present invention;

FIG. 4B is a cross-sectional view of the first connection element of FIG. 4A according to an embodiment of the present invention;

FIG. 5A is a schematic, perspective view of a first contact pin according to an embodiment of the present invention;

FIG. 5B is a schematic, perspective view of the first contact pin of FIG. 5A engaged with lead or lead extension conductor according to an embodiment of the present invention;

FIG. 6A is a schematic, perspective view of a second connection element according to an embodiment of the present invention;

FIG. 6B is a cross-sectional view of the second connection element of FIG. 6A according to an embodiment of the present invention;

FIG. 7A is a schematic, perspective view of a crimpable contact according to an embodiment of the present invention;

FIG. 7B is a schematic, perspective view of the crimpable contact of FIG. 7A engaged with a sleeve contact according to an embodiment of the present invention;

FIG. 8 is a cross-sectional view of the connector of FIG. 3 according to an embodiment of the present invention; and

FIG. 9 is a schematic diagram of an electrical stimulation system according to an embodiment of the present invention.

### DETAILED DESCRIPTION

The present invention is directed to the area of implantable electrical stimulation systems and methods of making and using the systems. The present invention is also directed to implantable micro extension connectors for electrical stimulation devices, as well as methods of making and using the same.

Suitable implantable electrical stimulation systems include, but are not limited to, a least one lead with one or more electrodes disposed along a distal end of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,203,548; 7,244,150; 7,450,997; 7,596,414; 7,610,103; 7,672,734;7,761,165; 7,783,359; 7,792,590; 7,809,446; 7,949,395; 7,974,706; 6,175,710; 6,224,450; 6,271,094; 6,295,944; 6,364,278; and 6,391,985; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; and 2013/0105071; and U.S. patent applications Ser. Nos. 12/177,823 and 13/750,725, all of which are incorporated by reference in their entireties.

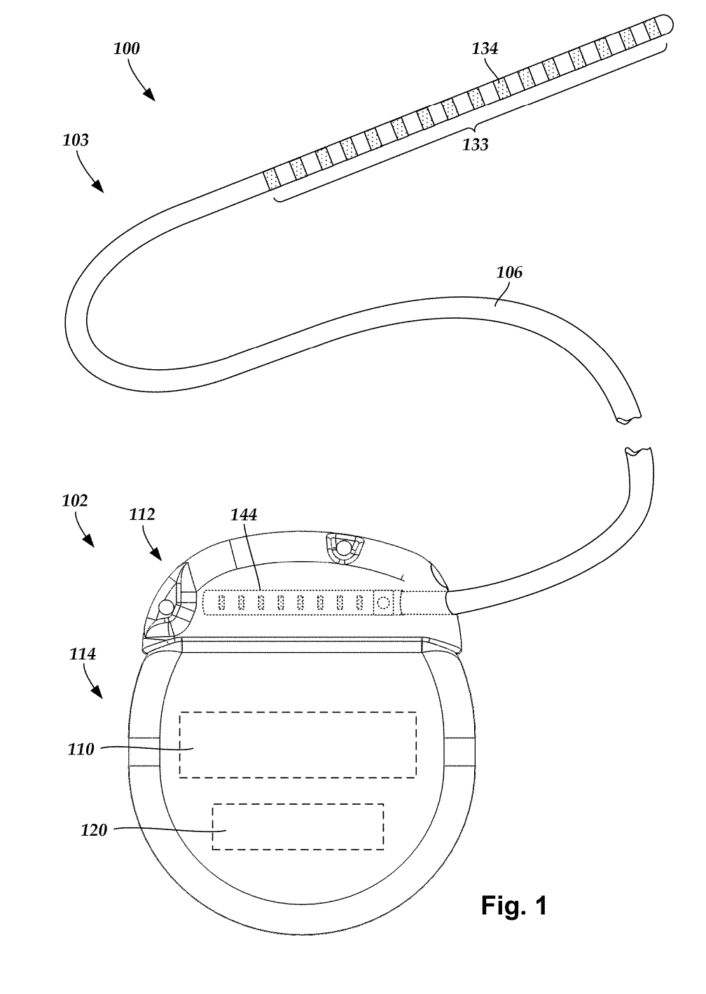

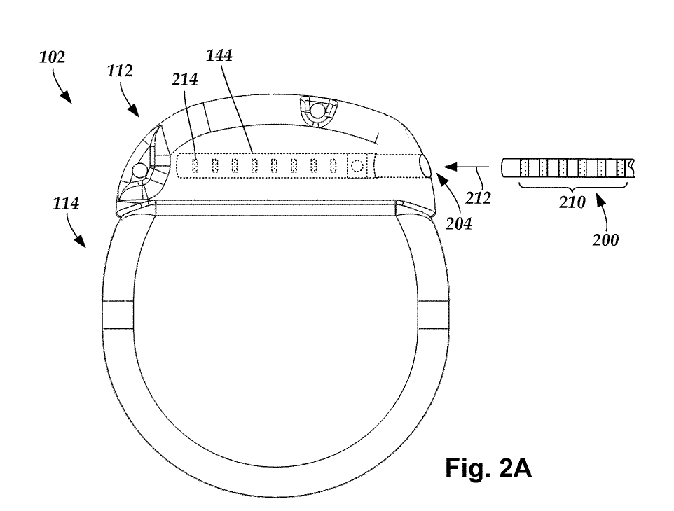

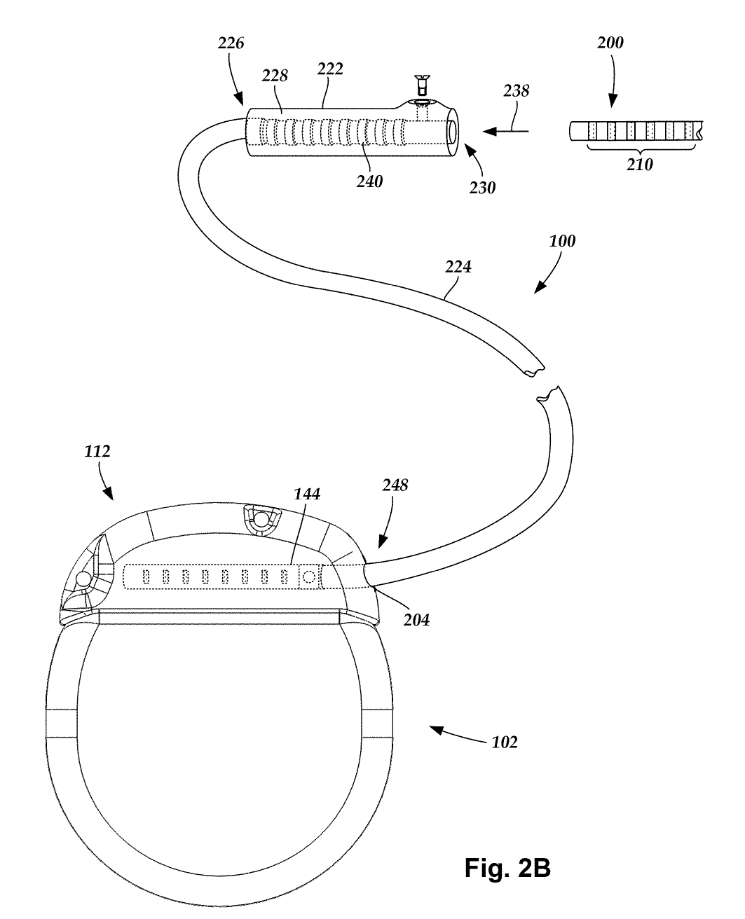

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes one or more lead bodies 106, an array of electrodes 133, such as electrode 134, and an array of terminals (e.g., 210 in FIG. 2A-2B) disposed along the one or more lead bodies 106. In at least some embodiments, the lead is isodiametric along a longitudinal length of the lead body 106.

The lead 103 can be coupled to the control module 102 in any suitable manner. In at least some embodiments, the lead 103 couples directly to the control module 102. In at least some other embodiments, the lead 103 couples to the control module 102 via one or more intermediate devices (200 in FIGS. 2A-2B). For example, in at least some embodiments one or more lead extensions 224 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

The control module 102 typically includes a connector housing 112 and a sealed electronics housing 114. Stimulation circuitry 110 and an optional power source 120 are disposed in the electronics housing 114. A control module connector 144 is disposed in the connector housing 112. The control module connector 144 is configured and arranged to make an electrical connection between the lead 103 and the stimulation circuitry 110 of the control module 102.

The electrical stimulation system or components of the electrical stimulation system, including the lead body 106 and the control module 102, are typically implanted into the body of a patient. The electrical stimulation system can be used for a variety of applications including, but not limited to, brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. The number of electrodes 134 in each array 133 may vary. For example, there can be two, four, six, eight, ten, twelve, fourteen, sixteen, or more electrodes 134. As will be recognized, other numbers of electrodes 134 may also be used.

The electrodes of the lead body 106 are typically disposed in, or separated by, a non-conductive, biocompatible material such as a polymeric or elastomeric material, for example, silicone, polyurethane, polyetheretherketone (“PEEK”), epoxy, and the like or combinations thereof. The lead body 106 may be formed in the desired shape by any process including, for example, molding (including injection molding), casting, and the like. The non-conductive material typically extends from the distal end of the lead body 106 to the proximal end of the lead body 106.

Terminals (e.g., 210 in FIGS. 2A-2B) are typically disposed along the proximal end of the lead body 106 of the electrical stimulation system 100 (as well as any splitters, lead extensions, adaptors, or the like) for electrical connection to corresponding connector contacts (e.g., 214 and 240 in FIG. 2B). The connector contacts are disposed in connectors (e.g., 144 in FIGS. 1-2B; and 222 in FIG. 2B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the lead body 106, for example, for inserting a stylet to facilitate placement of the lead body 106 within a body of a patient. Additionally, there may be one or more lumens (not shown) that open at, or near, the distal end of the lead body 106, for example, for infusion of drugs or medication into the site of implantation of the lead body 106. In at least one embodiment, the one or more lumens are flushed continually, or on a regular basis, with saline, epidural fluid, or the like. In at least some embodiments, the one or more lumens are permanently or removably sealable at the distal end.

FIG. 2A is a schematic side view of one embodiment of a proximal end of one or more elongated devices 200 configured and arranged for coupling to one embodiment of the control module connector 144. The one or more elongated devices may include, for example, the lead body 106, one or more intermediate devices (e.g., the lead extension 224 of FIG. 2B, an adaptor, or the like or combinations thereof), or a combination thereof.

The control module connector 144 defines at least one port into which a proximal end of the elongated device 200 can be inserted, as shown by directional arrow 212. In FIG. 2A (and in other figures), the connector housing 112 is shown having one port 204. The connector housing 112 can define any suitable number of ports including, for example, one, two, three, four, five, six, seven, eight, or more ports.

The control module connector 144 also includes a plurality of connector contacts, such as connector contact 214, disposed within each port 204. When the elongated device 200 is inserted into the port 204, the connector contacts 214 can be aligned with a plurality of terminals 210 disposed along the proximal end(s) of the elongated device(s) 200 to electrically couple the control module 102 to the electrodes (134 of FIG. 1) disposed at a distal end of the lead 103. Examples of connectors in control modules are found in, for example, U.S. Pat. Nos. 7,244,150 and 8,224,450, which are incorporated by reference.

FIG. 2B is a schematic side view of another embodiment of the electrical stimulation system 100. The electrical stimulation system 100 includes a lead extension 224 that is configured and arranged to couple one or more elongated devices 200 (e.g., the lead body 106, an adaptor, another lead extension, or the like or combinations thereof) to the control module 102. In FIG. 2B, the lead extension 224 is shown coupled to a single port 204 defined in the control module connector 144. Additionally, the lead extension 224 is shown configured and arranged to couple to a single elongated device 200. In alternate embodiments, the lead extension 224 is configured and arranged to couple to multiple ports 204 defined in the control module connector 144, or to receive multiple elongated devices 200, or both.

A lead extension connector 222 is disposed on the lead extension 224. In FIG. 2B, the lead extension connector 222 is shown disposed at a distal end 226 of the lead extension 224. The lead extension connector 222 includes a connector housing 228. The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (134 of FIG. 1) disposed along the lead (103 in FIG. 1).

In at least some embodiments, the proximal end of the lead extension 224 is similarly configured and arranged as a proximal end of the lead 103 (or other elongated device 200). The lead extension 224 may include a plurality of electrically conductive wires (not shown) that electrically couple the connector contacts 240 to a proximal end 248 of the lead extension 224 that is opposite to the distal end 226. In at least some embodiments, the conductive wires disposed in the lead extension 224 can be electrically coupled to a plurality of terminals (not shown) disposed along the proximal end 248 of the lead extension 224. In at least some embodiments, the proximal end 248 of the lead extension 224 is configured and arranged for insertion into a connector disposed in another lead extension (or another intermediate device). In other embodiments (and as shown in FIG. 2B), the proximal end 248 of the lead extension 224 is configured and arranged for insertion into the control module connector 144.

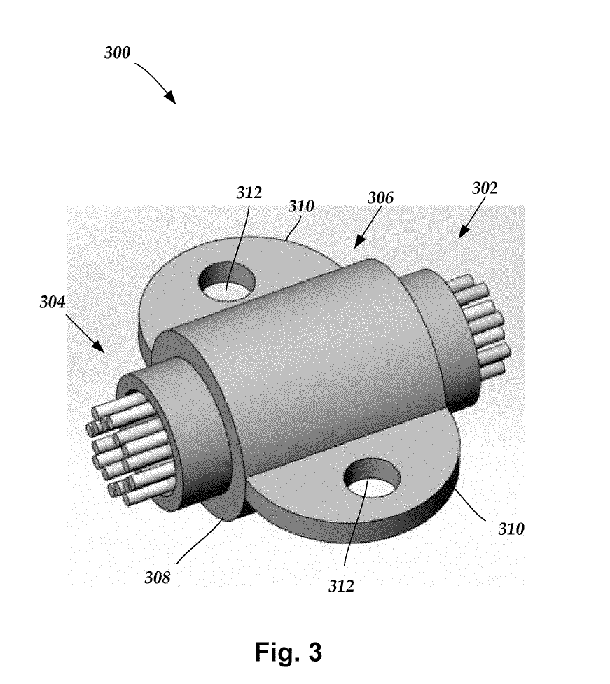

FIGS. 3A and 3B illustrate, schematically, a lead extension connector 300, which may take the place of the lead extension connector 222 (FIG. 2B). In at least some embodiments, the connector 300 includes a first connection element 302, a second connection element 304, and a coupler 306. The connector 300 permits the elongated device 200 (FIG. 2B) to be electrically coupled to a lead extension 224 (FIG. 2B). The various components of the connector 300 may be made using machining or micromachining processes such as, but not limited to, three-dimensional (3D) printing, crimping, molding, insert molding, soldering, casting, laser cutting, etching, and water jet cutting. In at least some embodiments, the connector 300 provides a modular lead technology that may allow standardized connectors such that arrays can be built independent of the lead body and the lead body to be built independent of the lead.

In at least some embodiments, the coupler 306 takes the form of an internally threaded cylindrical body 308 that urges the first connection element 302 into a sealing contact with the second connection element 304. The coupler 306 optionally includes wing sections 310 that extend radially outward. In at least some embodiments, the wing sections 310 extend in opposite directions from each other (e.g., one-hundred and eighty degrees opposite) relative to the cylindrical body 308. At least one of the wing sections 310 includes a suture hole 312 for anchoring or otherwise attaching the connector 300, via sutures (not shown), to a patient's tissue.

Conventional lead extensions may be large and bulky. In both (spinal cord stimulators) SCSs and (deep brain stimulators) DBSs, there is a desire to increase conductor (e.g., contact) count. New lead designs and higher resolution stimulation may have at least sixteen (16) contacts. In at least some embodiments, the connector 300 may advantageously reduce the size and length of the connection between a proximal end of a lead and a distal end of a lead extension while providing for a higher density of contacts, for example an arrangement of at least sixteen contacts. According to at least some embodiments, the connector 300 functions as a low profile, small connector having at least sixteen contacts that may be used for SCS, DBS or both.

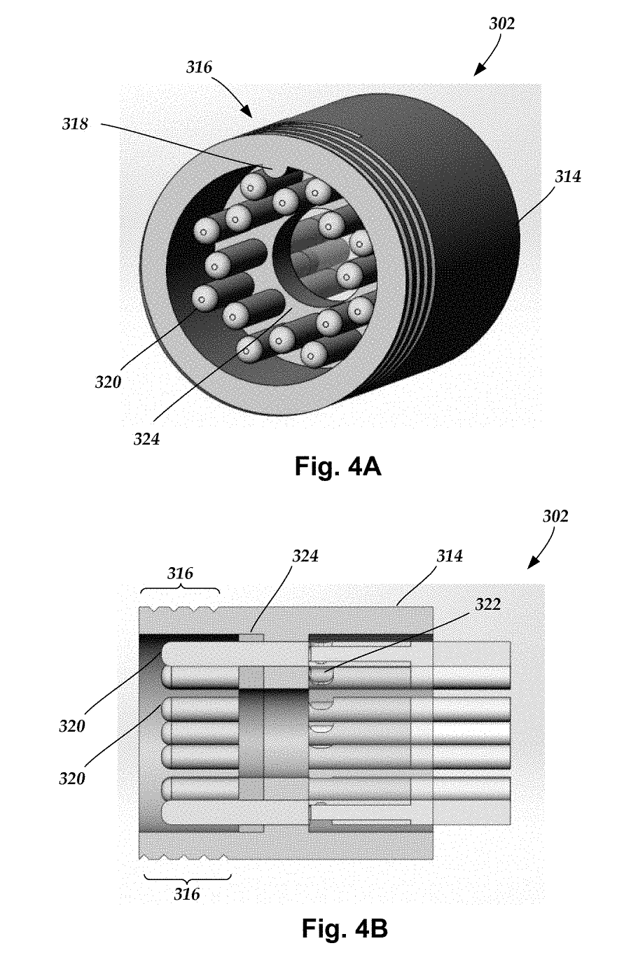

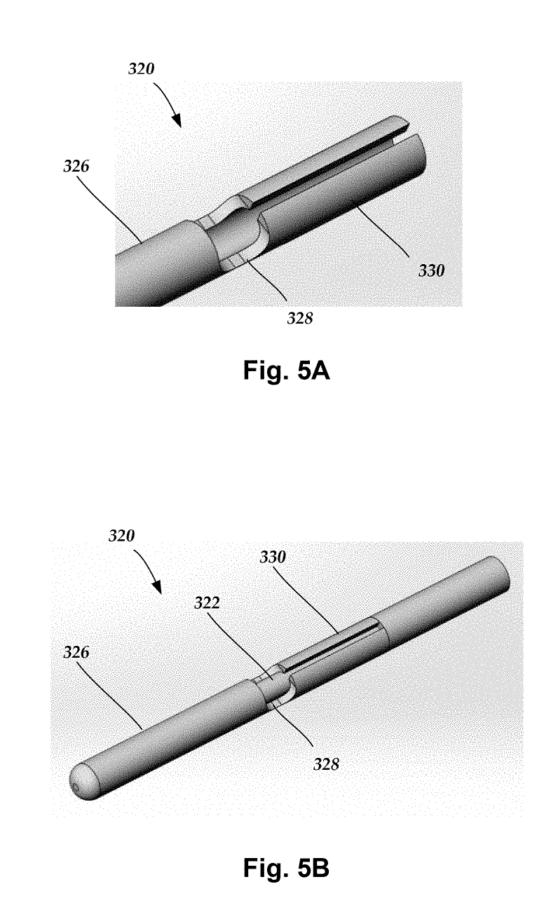

FIGS. 4A through 5B illustrate, schematically, the first connection element 302 having a first housing 314 and first external threads 316 (i.e., threads machined into an external surface of the first housing 314). In at least some embodiments, the first housing 314 includes an integrally formed or attached first keying feature 318, for example, the keying feature 318 may take the form of a rounded protuberance extending from an interior surface of the first housing 314. Alternatively, the first keying feature 318 may take the form of a channel or groove formed into the first housing 314. The first keying feature 318 allows the first connection element 302 to be aligned for engagement with the second connection element 304. Within the first housing 314, an array of first contacts 320 for coupling to lead conductors 322 (FIG. 5B) are at least partially embedded or disposed in a first sealing member 324, which is made of a non-conductive material. The first housing 314 may include a flange or shoulder that the first sealing member 324 abuts against. By way of example, the material for the first sealing member 324 may compressive or springy (i.e., elastomeric) and capable of forming a seal when urged or pressed against another component. The first sealing member may also be made from a biocompatible material such as an elastomeric or polymeric material, for example, silicone, polyurethane, PEEK, epoxy, and the like or combinations thereof.

Referring to FIGS. 5A and 5B, first lead conductors 322 are inserted into and crimped to first contact pins 320. The first lead conductors 322 may be associated with a lead body or a lead extension body. In at least some embodiments, the first contact pins 320 (FIG. 5A) include a receiving portion 326, a relief portion 328 and a crimpable portion 330. As illustrated in FIG. 5B, the first lead conductors 322 are inserted into the first contact pins 320 and secured thereto by crimping or otherwise squeezing the crimpable portions 330 onto the first lead conductors 322. The first contact pins 320 can be made from an electrically conductive material such as, but not limited to, a platinum iridium material. In at least some other embodiments, the first lead conductors 322 can be secured to the first contact pins 320 by welding, soldering or another assembly technique.

Referring back to FIG. 4B, the first sealing member 324 may be pressed or otherwise inserted into the first housing 314. After all of the lead conductors 322 are secured to the respective first contact pins 320, the conductor-pin assemblies are inserted into the first housing 314 and pressed into the first sealing member 324. The first contact pins 320 extend from the first sealing member 324.

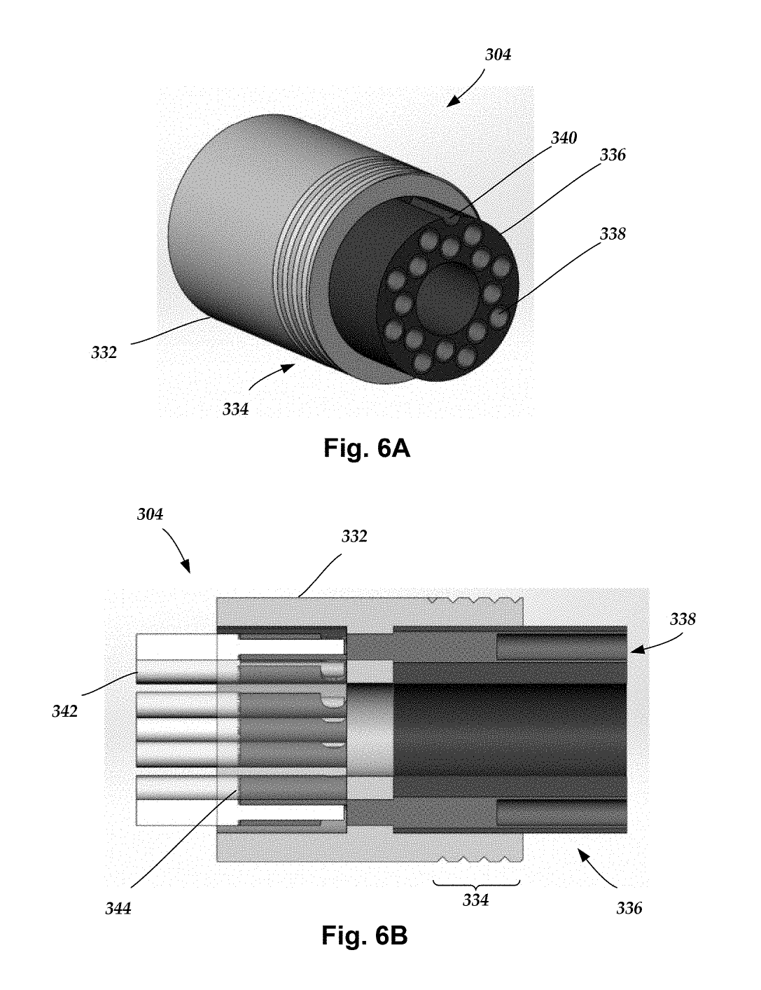

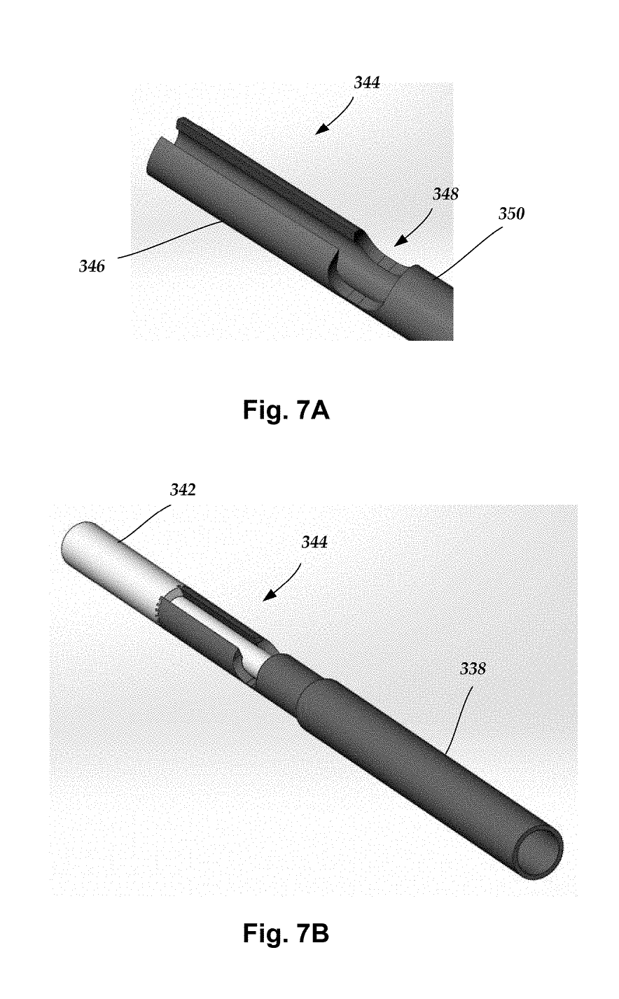

FIGS. 6A through 7B illustrate, schematically, the second connection element 304 having a second housing 332 and second external threads 334 (i.e., threads machined into an external surface of the second housing 332). A second sealing member 336 extends from the second housing 332. The second sealing member 336 includes a plurality of electrically conductive sleeve contacts 338 that are disposed or embedded within the second sealing member 336. The second sealing member 336 can be made of a non-conductive material such as, but not limited to, a biocompatible material such as an elastomeric or polymeric material, for example, silicone, polyurethane, PEEK, epoxy, and the like or combinations thereof. In addition, the second sealing member may also be compressive or springy (e.g., elastomeric) and capable of forming a seal when urged or pressed against another component. In at least some embodiments, the second sealing member 336 includes a molded or machined second keying feature 340, for example, the keying feature 340 may take the form of a rounded channel or groove recessed into an outer surface of the second sealing member 336. Alternatively, the second keying feature 340 may take the form of a protuberance in which the first keying feature 318 (FIG. 4A) would then take the form of a channel or groove. The first and second keying features 318, 340 are complementarily shaped with respect to one another. The second keying feature 340 cooperates with the first keying feature 318 (FIG. 4A) to allow the first and second connection elements 302, 304 to be aligned for engagement.

Referring to FIGS. 7A and 7B, second lead conductors 342 are inserted into and crimped to crimp contacts 344. In at least some embodiments, each crimp contact 344 (FIG. 7A) includes a crimpable portion 346, a relief portion 348 and a sleeve engagement portion 350. As illustrated in FIG. 7B, the second lead conductors 342 are inserted into the crimp contacts 344 and secured thereto by crimping or otherwise squeezing the crimpable portions 346. The crimp contacts 344 are inserted into the sleeve contacts 338, which may already be disposed in the second sealing member 336 or may be inserted as assembled with the second lead conductors 342 and the crimp contacts 344. The sleeve contacts 338 and the crimp contacts 342 can be made from an electrically conductive material such as, but not limited to, a platinum iridium material. In at least some other embodiments, the second lead conductors 342 can be secured to the sleeve contacts 338 by welding, soldering or another assembly technique.

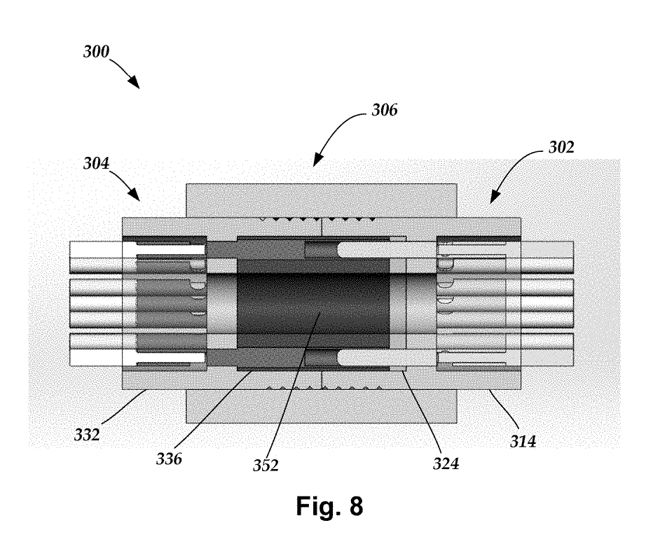

FIG. 8 shows a cross-sectional view of the connector 300. In one embodiment, the coupler 306 may be threaded onto the first connection element 302 and the second connection element 304 is threaded into the coupler 306. In this embodiment, the second housing 332 is free spinning relative to the second sealing member 336, thus allowing the second housing 332 to be threaded into the wing nut coupler 306.

In another embodiment, the coupler 306 may be threaded onto the second connection element 304 and the first connection element 302 is threaded into the coupler 306. In this embodiment, the first housing 314 is free spinning relative to the first sealing member 324, thus allowing the first housing 314 to be threaded into the wing nut coupler 306.

The coupler 306 will typically be attached, permanently or temporarily, to one of the first or second connection elements 302, 304. In such an assembly, the coupler 306 will be free spinning relative to at least the connection element to which it is not attached. The coupler 306 operates to urge the first and second sealing members 324, 336 together to form a fluid resistant seal around the conductors and contacts. Either or both of the first and second sealing members 324, 336 may be compressible to form a fluid resistant seal within the connector 300. It is appreciated that the connector may have an operational life, which may vary depending on the environment or manner in which it is employed, and preferably the fluid resistant seal will minimize or reduce interaction between the conductors/contacts and the bodily fluid for at least the operational life.

The connector 300 includes a central lumen 352 that extends through both the first and second sealing members 324, 336. The central lumen 352 permits a stylet or other instrument to be inserted through the connector 300.

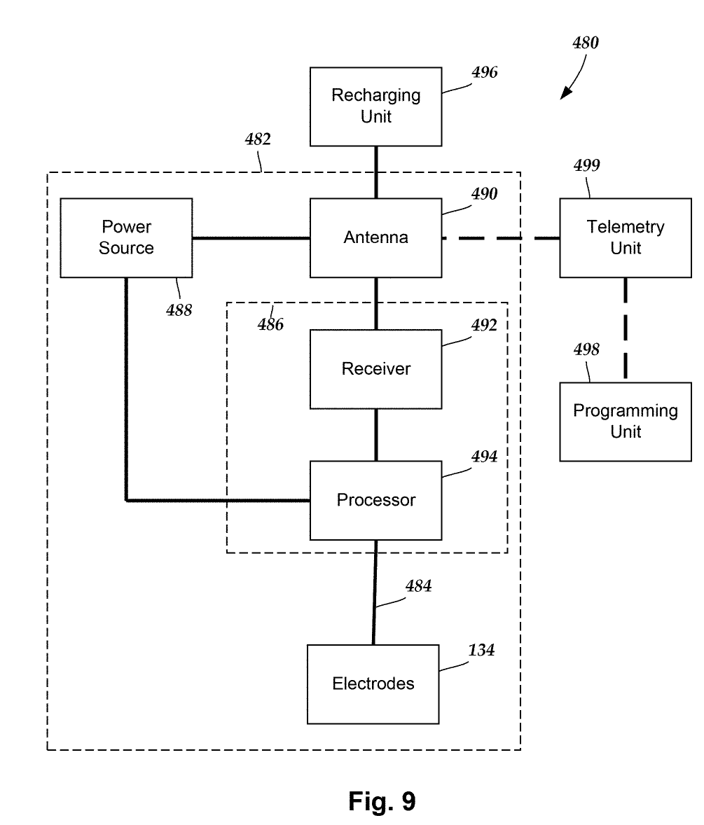

FIG. 9 is a schematic overview of one embodiment of components of an electrical stimulation arrangement 480 that includes an electrical stimulation system 482 with a lead 484, stimulation circuitry 486, a power source 488, and an antenna 490. The electrical stimulation system can be, for example, any of the electrical stimulation systems described above. It will be understood that the electrical stimulation arrangement can include more, fewer, or different components and can have a variety of different configurations including those configurations disclosed in the stimulator references cited herein.

If the power source 488 is a rechargeable battery or chargeable capacitor, the power source may be recharged/charged using the antenna 490, if desired. Power can be provided for recharging/charging by inductively coupling the power source 488 through the antenna 490 to a recharging unit 496 external to the user. Examples of such arrangements can be found in the references identified above.

In one embodiment, electrical current is emitted by the electrodes (such as electrodes 134 in FIG. 1) on the lead 484 to stimulate nerve fibers, muscle fibers, or other body tissues near the electrical stimulation system. The stimulation circuitry 486 can include, among other components, a processor 494 and a receiver 492. The processor 494 is generally included to control the timing and electrical characteristics of the electrical stimulation system. For example, the processor 494 can, if desired, control one or more of the timing, frequency, strength, duration, and waveform of the pulses. In addition, the processor 494 can select which electrodes can be used to provide stimulation, if desired. In some embodiments, the processor 494 selects which electrode(s) are cathodes and which electrode(s) are anodes. In some embodiments, the processor 494 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 498 that, for example, allows modification of pulse characteristics. In the illustrated embodiment, the processor 494 is coupled to a receiver 492 which, in turn, is coupled to the antenna 490. This allows the processor 494 to receive instructions from an external source to, for example, direct the pulse characteristics and the selection of electrodes, if desired.

In one embodiment, the antenna 490 is capable of receiving signals (e.g., RF signals) from an external telemetry unit 499 that is programmed by the programming unit 498. The programming unit 498 can be external to, or part of, the telemetry unit 499. The telemetry unit 499 can be a device that is worn on the skin of the user or can be carried by the user and can have a form similar to a pager, cellular phone, or remote control, if desired. As another alternative, the telemetry unit 499 may not be worn or carried by the user but may only be available at a home station or at a clinician's office. The programming unit 498 can be any unit that can provide information to the telemetry unit 499 for transmission to the electrical stimulation system 482. The programming unit 498 can be part of the telemetry unit 499 or can provide signals or information to the telemetry unit 499 via a wireless or wired connection. One example of a suitable programming unit is a computer operated by the user or clinician to send signals to the telemetry unit 499.

The signals sent to the processor 494 via the antenna 490 and the receiver 492 can be used to modify or otherwise direct the operation of the electrical stimulation system 482. For example, the signals may be used to modify the pulses of the electrical stimulation system such as modifying one or more of pulse duration, pulse frequency, pulse waveform, and pulse strength. The signals may also direct the electrical stimulation system 482 to cease operation, to start operation, to start charging the battery, or to stop charging the battery.

Optionally, the electrical stimulation system 482 may include a transmitter (not shown) coupled to the processor 494 and the antenna 490 for transmitting signals back to the telemetry unit 499 or another unit capable of receiving the signals. For example, the electrical stimulation system 482 may transmit signals indicating whether the electrical stimulation system 482 is operating properly or not or indicating when the battery needs to be charged or the level of charge remaining in the battery. The processor 494 may also be capable of transmitting information about the pulse characteristics so that a user or clinician can determine or verify the characteristics.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation system comprising:
a lead having a plurality of lead conductors;
a plurality of electrodes located along a distal portion of the lead, the plurality of electrodes electrically coupled to the plurality of lead conductors;
a lead extension having a plurality of lead extension conductors;
a plurality of terminals located along a proximal portion of the lead extension, the plurality of terminals electrically coupled to the lead extension;
a first connection element having a first sealing member and a plurality of first contacts at least partially disposed within the first sealing member; and
a second connection element having a second sealing member and a plurality of second contacts at least partially disposed within the second sealing member;
wherein the lead is coupled to one of the first or second connection elements, the plurality of first contacts are electrically coupled to a one of the lead conductors or the lead extension conductors,
wherein the lead extension is coupled to another one of the first or second connection elements, the plurality of second contacts are electrically coupled to another one of the lead conductors or the lead extension conductors,
wherein the first and second connection elements electrically couple the lead conductors with the lead extension conductors; and
a coupler engageable with the first and second connection elements to urge the first and second sealing members together to resist bodily fluid interaction with the lead conductors, the lead extension conductors, the first contacts, or the second contacts.

2. The electrical stimulation system of claim 1, further comprising a central lumen extending through the first and second sealing members.

3. The electrical stimulation system of claim 1, wherein the first and second housings include complementary keying features.

4. The electrical stimulation system of claim 1, wherein at least one of the first or second contacts are crimpable contacts.

5. The electrical stimulation system of claim 1, wherein the first housing is free to rotate relative to the first sealing member.

6. The electrical stimulation system of claim 1, wherein the second housing is free to rotate relative to the second receiving member.

7. The electrical stimulation system of claim 1, wherein the conductors and contacts are made from a platinum iridium material.

8. The electrical stimulation system of claim 1, wherein the first and second sealing members are made from an elastomeric material.

9. The electrical stimulation system of claim 1, wherein the coupler includes wing portions that extend from the coupler.

10. The electrical stimulation system of claim 9, wherein at least one of the wing portions includes a suture hole.

11. The electrical stimulation system of claim 1, further comprising a control module having a housing and an electronic subassembly disposed in the housing, wherein the lead extension is electrically coupleable to the electronic subassembly.

12. A connector comprising:
a first connection element having a first housing and a first non-conductive, elastomeric sealing member at least partially disposed within the first housing, wherein the first housing includes a first outer surface and first machine threads formed on the first outer surface;
a plurality of male contacts at least partially disposed within the first sealing member;
a second connection element having a second housing and a second, non-conductive, elastomeric sealing member extending from the second housing, wherein the second housing includes a second outer surface and second machine threads formed on the second outer surface;
a plurality of female contacts at least partially disposed within the second sealing member; and
a coupler having a first end portion and a second end portion, the first end portion having first internal threads for engaging the first machine threads, the second end portion having second internal threads for engaging the second machine threads, wherein engagement of the coupler with the first and second connection elements urges the first and second sealing members together to resist bodily fluid from interacting with the male contacts, the female contacts, or both.

13. The connector of claim 12, wherein the first housing is free to rotate relative to the first sealing member.

14. The connector of claim 12, wherein the second housing is free to rotate relative to the second sealing member.

15. The connector of claim 12, further comprising a central lumen extending through the first and second sealing members.

16. The connector of claim 12, wherein the first and second housings include complementary keying features.

17. The connector of claim 12, wherein the first and second sealing members are made from a polymeric material.

18. The connector of claim 12, wherein the coupler includes wing portions that extend from the coupler.

19. The connector of claim 18, wherein at least one of the wing portions includes a suture hole.

20. A method of stimulating patient tissue, the method comprising:
implanting the electrical stimulation system of claim 1 into the patient tissue; and
controllably modulating the plurality of electrodes with electrical pulses from a control module to stimulate the patient tissue.
